SEQUENCE LISTING (15 sequences)

SEQ ID NO: 1 - DNA, 23 bp, Artificial Sequence
  Primer PLU F
gctctttcttgatttcttggatg

SEQ ID NO: 2 - DNA, 21 bp, Artificial Sequence
  Primer PLU R
agcaggttaagatctcgttcg

SEQ ID NO: 3 - DNA, 20 bp, Artificial Sequence
  Primer PLU P
atggccgtttttagttcgtg

SEQ ID NO: 4 - DNA, 27 bp, Artificial Sequence
  Primer FAL F
attgcttttgagaggttttgttacttt

SEQ ID NO: 5 - DNA, 29 bp, Artificial Sequence
  Primer FAL R
gctgtagtattcaaacacaatgaactcaa

SEQ ID NO: 6 - DNA, 20 bp, Artificial Sequence
  Primer FAL P
cataacagacgggtagtcat

SEQ ID NO: 7 - DNA, 23 bp, Artificial Sequence
  Primer VIV F
gcaacgcttctagcttaatccac

SEQ ID NO: 8 - DNA, 22 bp, Artificial Sequence
  Primer VIV R
caagccgaagcaaagaaagtcc

SEQ ID NO: 9 - DNA, 20 bp, Artificial Sequence
  Primer VIV P
actttgtgcgcattttgcta

SEQ ID NO: 10 - DNA, 21 bp, Artificial Sequence
  Primer RNaseP F
tgtttgcagatttggacctgc

SEQ ID NO: 11 - DNA, 20 bp, Artificial Sequence
  Primer RNaseP R
aatagccaaggtgagcggct

SEQ ID NO: 12 - DNA, 16 bp, Artificial Sequence
  Primer RNaseP P
tgcgcggacttgtgga

SEQ ID NO: 13 - DNA, 27 bp, Artificial Sequence
  Primer IC F
aaagaaactaggagagatgtggaacaa

SEQ ID NO: 14 - DNA, 21 bp, Artificial Sequence
  Primer IC R
agcttggcagctttcttctca

SEQ ID NO: 15 - DNA, 25 bp, Artificial Sequence
  Primer IC P
actgcagcagatgacaagcagccct

(19) United States
(12) Patent Application Publication
(10) Pub. No.: US 2015/0232950 A1
(43) Pub. Date: Aug. 20, 2015

(54) Sensitive Multiplex QPCR Assay For The Detection of Malaria

(71) Applicant: United States, as represented by the Secretary of the Army, Fort Detrick, MD (US)

(72) Inventors: Edwin Kamau, DPO (AE); Christian F. Ockenhouse, Chevy Chase, MD (US); Karla C. Feghali, McLean, VA (US); Saba Alemayehu, Silver Springs, MD (US)

(21) Appl. No.: 14/625,190

(22) Filed: Feb. 18, 2015

Related U.S. Application Data
(60) Provisional application No. 61/941,279, filed on Feb. 18, 2014.

(51) Int. Cl. C12Q 1/68

(52) U.S. Cl. CPC C12Q 1/6893; C12Q 2600/16; C12Q 2600/158

(57) ABSTRACT

The disclosure provides a multiplex real-time PCR method for determining the absolute quantification of Plasmodium parasites within a sample, and in some instances, as a measure of parasites/μl. Further, the disclosure provides a method of determining the strain of malaria within a sample.

### RELATED APPLICATIONS

This application makes reference to and claims priority to U.S. Provisional Application Ser. No. 61/941,279 filed on Feb. 18, 2014, which is hereby incorporated herein by reference in its entirety.

### FEDERALLY SPONSORED RESEARCH OR DEVELOPMENT

The present invention was made with support from the United States Government through the Walter Reed Army Institute of Research. The United States government has certain rights in this invention.

### BACKGROUND

Malaria remains one of the most burdensome and lethal infectious diseases in tropical and sub-tropical countries. Currently, microscopy and rapid diagnostic tests are used for the detection of malaria. Despite some gains made in diagnosis of malaria through use of molecular methods, microscopy remains the gold standard technique for diagnosis and quantification of malaria, evaluation of clinical trials efficacy, and epidemiological surveys. However, these assays are inaccurate, have low sensitivity, and cannot be utilized in high throughput applications. Further, microscopy is also limited by the need for the extensive user training, variability between microscopists, and difficulty in certifying results [1], [2]. In expert hands, microscopy has a detection limit of 10 to 50 parasites/μL [28], [29], but the average microscopist typically works near a detection limit of about 100 parasites/μL, which limits the use of microscopy in identification of subjects having a low parasite burden. [30]. Studies have shown that mosquitoes do get infected at submicroscopic levels, and can transmit malaria [31], [32]. Thus, current methods of detection are labor intensive and are not sensitive enough to detect sub-clinical cases.

Because the threshold for fever and clinical disease for both P. falciparum and P. vivax malaria is about 10 parasites/μl blood, new and reliable diagnostic tests that are ultrasensitive and specific for malaria are needed to replace the current gold standard microscopy test, the Giemsa-stained thick blood smear. The detection of parasites in subjects sub-clinically infected with either P. falciparum or P. vivax malaria is useful in many respects to help combat infection by malaria. For example, subclinical detection can detect infection before clinical symptoms develop and thus provide treatment earlier than the standard methods. This in turn can reduce the spread of malaria by treating asymptomatic patients and can stop the infection cycle between vector mosquitoes and the asymptomatic carriers. Subclinical detection can also be useful in assessing the efficacy of malaria vaccines or to evaluate new antimalarial drugs. Further, concomitant use of a molecular-based assay for detecting Plasmodium parasites would be an excellent safeguard against possible false-negative results arising from diagnosis by expert microscopists [33].

Therefore, in an era of malaria control and elimination, highly sensitive methods with high throughput capabilities are needed for parasite detection and surveillance. Such methods can quantify the extent of submicroscopic infections and provide insight into the dynamics of malaria transmission.

Real-time PCR (rtPCR) can meet the requirements for utilization in malaria surveillance and epidemiological studies. Although rtPCR methodology can include similar steps and protocols, differences in reagents, standards used for quantification, dilution ranges, instruments or platforms, assay analysis methods, data interpretations, and the like, can contribute to differences in the reported detection limits. Currently, no PCR-based assay has been validated or approved by the FDA for the detection and quantification of malaria. While this may be due to any number of factors such as, for example, the lack of consensus or standardized methods in performing qPCR, it remains difficult to evaluate and/or compare the quality of work reported by different studies and/or cross-platform assay analysis. See, e.g., Alemayehu et al. Malaria Journal 2013, 12:277.

While qPCR is a recognized technique, its application to methods for detecting plasmodium and particularly detection at sub-clinical levels has been plagued by inconsistent results and lack of uniform techniques. Thus, its potential use in the field remains unfulfilled.

### SUMMARY

In an aspect, the disclosure relates to a method of quantifying the amount of one or more Plasmodium species in a sample comprising:

(a) amplifying in parallel:
(i) a target nucleic acid sequence from the one or more Plasmodium species;
(ii) a standard nucleic acid sequence of a predetermined concentration corresponding to the one or more Plasmodium species;

(b) detecting the amount of the (i) target nucleic acid sequence and (ii) standard nucleic acid sequence amplified in (a); and
(c) quantifying the amount of the amplified target nucleic acid sequence by correlating the amount of the amplified target nucleic acid sequence detected in (b) to the amount of the amplified standard detected in (b).

In some aspects, step (a)(ii) comprises amplifying at least two or at least three different predetermined concentrations of the standard nucleic acid sequence. In some aspects, step (a)(i) and (a)(ii) comprise using primers found in Table 1. In some further aspects, the method includes extracting DNA from the sample prior to step (a).

In another aspect, the disclosure provides a method of identifying two or more Plasmodium species in a sample comprising:

(a) amplifying in parallel:
(i) a first target nucleic acid sequence from a first Plasmodium species and a second target sequence from an at least one additional Plasmodium species, when at least two Plasmodium species are present in the sample;
(ii) a first standard nucleic acid sequence and at least one additional standard nucleic acid sequence of a predetermined concentration, wherein the first and at least one additional standards correspond to the first and the at least one additional Plasmodium species of (i), respectively;
(b) detecting the amount of the (i) first and at least one additional target nucleic acid sequence and (ii) first and at least one additional standard nucleic acid sequence amplified in (a); and
(c) determining the presence of the first or the at least one additional Plasmodium species in the sample.

### BRIEF DESCRIPTION OF THE DRAWINGS

FIGS. 1A-1D: Titration of reaction master mix volume in qPCR reaction. Multiplex qPCR reactions were set-up that contained descending reaction master mix from 10 μl to 1 μl and 1 μl DNA template in each reaction. Experiments were performed in total replicates 8. All the four targets in the multiplex qPCR assay (PLU (1A), FAL (1B), VIV (1C), and RNaseP (1D)) were analyzed simultaneously. There was a negative correlation between reaction master mix volume and assay sensitivity. As the volume of the reaction master mix increased, the sensitivity of the qPCR decreased with 1 μl reaction master mix reaction being the most sensitive for PLU (FIG. 1A), FAL (FIG. 1B), and VIV (FIG. 1C) assays.

FIGS. 2A-2C. Linear regression plots for absolute qPCR assays. Real-time PCR assays were performed using plasmid DNAs for each assay: PLU assay (FIG. 2A), FAL assay (2B), and VIV assay (2C). Plasmid DNA was 10-fold serially diluted at each point and ran in 4-8 replicates. A linear regression plot was generated using GraphPad Prism. The slope, the Y-intercept and the r² value were determined. Data shown confirms that these assays performed with high efficiencies.

FIGS. 3A-3B. Analysis of parasite densities in clinical samples using absolute qPCR and microscopy. Absolute quantitative qPCR was performed using plasmid DNA as the standard to analyze clinical samples using the PLU assay (3A) and FAL assay (3B). The log 10 parasite densities in terms of parasite/μl was determined from qPCR assays and compared to the log 10 parasite densities as determined by expert microscopists. The correlation coefficient of parasite densities measured using the two methods was calculated using the nonparametric Spearman correlation coefficient. There was a statistically significant correlation between parasite density measured by microscopy and absolute quantitative qPCR.

FIGS. 4A-4B: Amplification plot showing LODs for detection of Plasmodium. To establish the LOD, standard 3D7 NA was serially 5-fold diluted from 2.00E4 to 4.10E-4 parasite/μl, and then qRT-PCR or qPCR was run using a genus-specific assay (4A and 4B). Amplification plots showing LODs by qRT-PCR using standard 3D7 NA. The lowest amplification for qRT-PCR was 2.05E-03 parasite/μl (4A), and for qPCR it was 5.23E-02 parasite/μl (4B). Delta Rn, magnitude of normalized fluorescence.

FIG. 5: Reproducibility of the genus-specific assay. Data are from serially diluted standard 3D7 NA assayed on different days by different operators, represented by different colors on the graph. The data show that the assay is highly reproducible.

FIGS. 6A-6B: Amplification plot showing LODs by qRT-PCR using a clinical sample that was serially 5-fold diluted. The lowest amplification for qRT-PCR was 6.61 E-03 parasite/μl (6A) and for qPCR was 2.97E-02 parasite/μl (6B).

FIG. 7: Addition of the reverse transcriptase enzyme to the qPCR assay increases sensitivity. The CT values of clinical samples were determined using qRT-PCR and qPCR. The difference in CT(ΔCT) for each clinical sample was determined and plotted against the parasite density as determined by a thick blood smear. An increase in the sensitivity of the assay by addition of the RT step was seen in all log groups.

FIGS. 8A-8B: Inclusion of the reverse transcriptase enzyme in the qPCR assay affects the quantification of parasites. The log parasite density of clinical samples determined by microscopy was compared to corresponding CT values obtained by qRT-PCR (8A) or qPCR (8B). There was a statistically significant correlation between parasite densities measured by microscopy and both PCR assays, with qPCR outperforming qRT-PCR.

### DETAILED DESCRIPTION

The inventors have identified methods that allow for the reproducible, ultra-low detection of Plasmodium species in biological samples which improve upon the currently existing microscopy methods. The methods can allow for specific identification of particular Plasmodium species in a sample. The methods also allow for detection of Plasmodium in a sample at levels that are lower than those associated with clinical manifestations of disease (e.g., before presentation of classic symptoms of malaria). The methods can be adapted for use in either singleplex or multiplex assay formats and can be used in high throughput assays. As discussed herein, the methods are unexpectedly specific and accurate for the detection and identification of Plasmodium at concentration levels that were previously not able to be reproducibly detected in a sample, which improves diagnosis and reproducibility in the clinical setting. Further, the methods allow for standardization of measurement of Plasmodium in samples and straightforward calculation of the quantity of any detected Plasmodium species in units of parasites/μl, the current unit used by microscopist for quantifying malaria in both subjects with or without clinical symptoms of malaria.

In one aspect, the disclosure relates to a method of quantifying the amount of one or more Plasmodium species in a sample. The method generally comprises (a) amplifying in parallel (i) a target nucleic acid sequence from the one or more Plasmodium species and (ii) a standard nucleic acid sequence of a predetermined concentration corresponding to the one or more Plasmodium species; (b) detecting the amount of the (i) target nucleic acid sequence and (ii) standard nucleic acid sequence amplified in (a); and (c) quantifying the amount of the amplified target nucleic acid sequence by correlating the amount of the amplified target nucleic acid sequence detected in to the amount of the amplified standard detected.

In some embodiments, the method comprises amplifying at least two different predetermined concentrations of the standard nucleic acid sequence. In some embodiments, the method can comprise amplifying at least three, four, five, or six different predetermined concentrations of the standard nucleic acid sequence. In some aspects, the amount of DNA in the predetermined concentrations of the standard concentrations is converted to the number of copies of Genomic Equivalence (GE) using DNA molecular weight. Embodiments of the method provide a standard curve that can be used to determine the quantity target of an unknown sample (e.g., linear regression or other curve-fitting methods).

In some embodiments, the method provides an absolute quantification of the amount of Plasmodium parasites in a sample from a subject by using a calibration curve where known amounts of external targets are amplified in a parallel group of reactions run under identical conditions to that of the samples.

The external targets may be standard molecules such as recombinant plasmid DNA carrying the target gene (plasmid DNA), genomic DNA or commercially synthesized oligonucleotide. In some embodiments, plasmid DNA is used. Suitable plasmid DNA can be made using any suitable primers directed to the sequence of interest, for example, the primers as described in Table 1. The PCR fragment amplified can be cloned into any suitable vector, for example, but not limited to, a TOPO® TA vectors (Life Sciences, Grand Island N.Y.).

In some embodiments, the absolute quantities of the standard DNA are determined by an alternative technique generally known in the art. Such techniques can include, for example, UV absorbance (OD₂₆₀) or fluorescent dye-binding methods. The concentration of the DNA is then converted to the number of copies or Genomic Equivalence [GE] using DNA molecular weight.

Absolute qPCR can be used to determine the quantity of the unknown target sequence based on the standards, such as by, for example, linear regression calculations. Absolute quantification has several advantages over relative quantification; it is highly reproducible, allows the generation of highly specific, sensitive and reproducible data [13]. It is a more precise approach for analyzing quantitative data and can be optimized and validated more readily than other quantitative methods. Absolute quantification of Plasmodium by qPCR has not been described previously.

Some embodiments provide an absolute quantitative multiplex qPCR assay for detection of Plasmodium spp., P. falciparum and P. vivax parasites. The absolute quantification is reported as parasites/μl, the same units as those used in microscopy.

In some aspects, the amplification of the target nucleic acid sequence and the standard nucleic acid sequence uses the primer pairs in Table 1. In some aspects, the nucleic acid sequence and the standard nucleic acid sequence using a primer pair specific for Plasmodium ssp., wherein the primers include a forward primer SEQ ID NO: 1 and reverse primer SEQ ID NO: 2 as shown in Table 1. In some aspects, a primer pair specific for P. falciparum is used, wherein the primers include a forward primer SEQ ID NO: 4 and reverse primer SEQ ID NO: 5 as shown in Table 1. In some aspects, a primer pair specific for P. vivax, wherein the primers include a forward primer SEQ ID NO: 7 and reverse primer SEQ ID NO: 8 as shown in Table 1 are used.

In some aspects, the method comprises amplifying an internal control to control for DNA extraction variations. Suitable internal controls include amplifying RNaseP as the internal control for the DNA extraction process using a primer pair specific for human RNaseP, wherein the primers include a forward primer SEQ ID NO: 10 and reverse primer SEQ ID NO: 11 shown in Table 1.

In some embodiments, a first target nucleic acid sequence from one Plasmodium species and a second target nucleic acid sequence from a second Plasmodium species are amplified in parallel. In some aspects, they are amplified in the same reaction vessel. The method further comprises amplifying in parallel a first standard nucleic acid sequence of at least one predetermined concentration corresponding to the first Plasmodium species and a second standard nucleic acid sequence of at least one predetermined concentration corresponding to the second Plasmodium species. In some aspects, the first Plasmodium species is P. falciparum and the second Plasmodium species is P. vivax.

In some aspects, the method comprises amplifying in parallel the target nucleic acid sequence and the standard nucleic acid sequence using a first primer pair specific for P. falciparum, wherein the primers include a forward primer SEQ ID NO: 4 and reverse primer SEQ ID NO: 5, and a second primer pair specific for P. vivax, wherein the primers include a forward primer SEQ ID NO: 7 and reverse primer SEQ ID NO: 8.

In some aspects, when one or more target is amplified in the same reaction vessel, the method further comprises using a probe for each target, wherein each probe comprises a unique reporter molecule that distinguishes the presence of each target present in the sample. Suitable probe sequences can be found in Table 1. Further, the unique reporter molecules can be selected from the group comprising fluorophores.

In reactions in which more than one target is amplified in a single reaction, probes are linked to unique reporter molecules that can be distinguished from each other. For example, suitable fluorophores can be used. Suitable fluorophores include, but are not limited to, CY-5, FAM, VIC, NED, and CY3. Suitable fluorophores are known in the art.

In some aspects, at least three target nucleic acid sequences are amplified and detected. In this aspect, the three targets are Plasmodium ssp., P. falciparum, and P. vivax. Suitable, in parallel three standard nucleic acid sequence of at least one predetermined concentration corresponding to the Plasmodium ssp., P. falciparum, and P. vivax are amplified. In some aspects, RNAseP is used in these reactions as an internal control. Amplifying RNaseP as an internal control for the DNA extraction process includes using a primer pair specific for human RNaseP, wherein the primers include a forward primer SEQ ID NO: 10 and reverse primer SEQ ID NO: 11. IN some aspects a probe is used to amplify and detect RNaseP, wherein the probe comprising, for example, SEQ ID NO: 12.

In some aspects, the methods further comprise converting the amount of the target nucleic acid sequence to a parasite/μl.

Malaria parasite density is expressed in terms of parasite/μl, based on Giemsa-staining of thick and/or thin blood smears. As such, it would be convenient for any qPCR assay that may supplement or replace microscopy as the standard diagnostic method, to be expressed in units of parasites/μl. Current qPCR assays use relative standard quantification methods to quantify parasite density in a sample where cultures or clinical samples with known parasite density are used. This method depends on accurate preparation of standard DNA for every assay and can involve growing cultures or obtaining a clinical sample quantified by an expert microscopist. Such procedures are inconvenient, time consuming, expensive and can be source of assay error. Thus, an absolute quantitative qPCR assay would present an advantage in accuracy and consistency relative to the current quantitative methods. Also, use of plasmid DNA can be produced in large quantities and if properly handed and stored, it can last for a long time. However, different structural types of standard DNA (circular versus linear) have been shown to affect the quantification and accuracy of qPCR assays. Activities that might affect the structure of plasmid DNA include freeze thawing, pipetting and vortexing. A recent study demonstrated that the linear DNA standards including linearized plasmids, but not the circular plasmid, are more reliable for absolute qPCR [13]. Therefore, in some embodiments, the plasmid DNA is linearized to improve their reliability. Plasmid DNA can be linearized by means known in the art, for example, but not limited to, enzyme digestion. The sensitivity of the assays in parasite/μl compared well when using either plasmid DNA or genomic DNA. In addition, there was a significant correlation between parasite densities and in terms of parasite/μl established using both methods. However, the average parasite density established using microscopy was higher compared to density established using qPCR, a phenomenon that have previously been observed [11]. To the best of our knowledge, this is the first time that plasmid DNA has been used in quantification of Plasmodium.

Most of the qPCR assays that have been described previously for detection of Plasmodium target the multicopy 18S ribosomal RNA (rRNA) genes [21]. Other targets such as mitochondrial genes, var and stevor genes have also been described [13,21]. These assays are designed as monoplex where they amplify a single target or as multiplex, where they amplify two targets or more. When used as a monoplex assay, the reported detection limit ranges from about 0.002 to 30 parasites/μL[14,21] whereas as a multiplex, the detection limit ranges from 0.2 to 5 parasites/μL [7,8,14,21]. Hermsen et al. [9] and Lee et al. [10] described some of the early monoplex qPCR assays for detection of Plasmodium spp with a detection limit of 0.02 parasites/μL and 0.1 parasites/μL respectively. Both of these studies targeted the rRNA genes. Recently, Farrugia et al. [13] described a monoplex assay that targets cytochrome b gene (ctyb) with a detection limit of 0.05 parasites/μL.

In some aspects, the disclosure provides a method of identifying two or more Plasmodium species in a sample comprising amplifying in parallel a first target nucleic acid sequence from a first Plasmodium species and a second target sequence from an at least one additional Plasmodium species; and a first and an at least one additional standard nucleic acid sequence of a predetermined concentration corresponding to the first and the at least one additional Plasmodium species. The method further comprises detecting the amount of the first and at least one additional target nucleic acid sequence and first and at least one additional standard nucleic acid sequence amplified and further determining the presence of the first or the at least one additional Plasmodium species in the sample. In some aspects, first Plasmodium species is P. falciparum and the at least one additional Plasmodium species is P. vivax. In some aspects, the at least two species are amplified in a single reaction vessel, and they are detected using unique probes linked to a reporter molecules. Suitable primers and probes are found in Table 1.

In one aspect, the disclosure provides a unique method that has the ability to differentiate in the same reaction vessel the type of malaria. Since there are four (4) human malarias, this assay allows the ability to differentiate the species in the human host in one reaction.

In some embodiments, the disclosure provides a multiplex PCR method for detection of malaria for clinical diagnosis. Other embodiments provide a multiplex PCR method for detection of malaria in donated blood. Yet other embodiments provide a multiplex PCR for malaria surveillance and epidemiological studies. Yet other embodiments provide multiplex PCR for malaria elimination and eradication campaign. Yet further embodiments provide a method for detection of subclinical levels of Plasmodium ssp. in a sample collected from a patient without clinical symptoms.

The present disclosure, in one aspect, provides a method that simultaneously detects at least two, for example, two or three Plasmodium targets and the human RNaseP gene as an endogenous control in a single reaction vessel. The method may be used to in vaccine and drug efficacy studies, for example, ongoing studies at Walter Reed Army Institute Research (WRAIR) in Silver Spring Md., Southeast Asia and in Africa.

Some embodiments describe a method that can detect at three different targets in a single reaction vessel and having equal to or greater sensitivity compared to a method of detecting each target in an individual reaction vessel. In some aspects, fluorophores were selected that had optimal compatibility and did not overlap in detection. The primer and probe sets disclosed in Table 1 performed with high efficiencies of more than 94%, high R2 values and very low STDEVs between replicates of each dilution. The qPCR method in some embodiments provides an acceptable PCR efficiency range is 100%±10% which is derived from a slope of −3.3±10%. It is to be understood that a reaction with lower efficiency will have lower sensitivity. For a PCR method to be considered 100% efficient, the CT difference between two successive concentrations in a 2-fold dilution is 1. To be able to quantify a 2-fold dilution in more than 99.7% of cases, the STDEV has to be ≦0.167. The examples provided herewith demonstrate that PCR chemistries in all reactions tested in Table 1 are robust and sensitive.

In some embodiments, the steps of PCR include DNA extraction from a sample, suitable a blood sample from a subject or patient. The quality of DNA obtained can impact all downstream activities. In routinely performed test(s), DNA extraction must be efficient, convenient and fast. Suitable methods of DNA extraction include, but are not limited to, mini-reparation of genomic DN or suitable kits used for purification of DNA, for example, Qiagen EZ1 DNA blood kit. Purification of DNA on EZ1 Advanced XL automated sample purification system takes 17-19 minutes to extract 14 samples using Qiagen EZ1 DNA blood kit. The quality of DNA obtained is the same as that obtained using manual kits. In some embodiments, automation reduces hands-on time which allows the technician to focus on other steps of setting up qPCR, improving on the overall time of getting results back from a run. It also improves the overall performance and handling of routine PCR assays.

In some embodiments, increasing the amount of blood volume extracted does not seem to improve sensitivity of the method of detection. For some embodiments, when a volume of more than 200 μl of blood was used for extraction and the DNA eluted in smaller volumes [such as start with 500 μl of blood and elute the DNA in 50-100 μl elution buffer], the sensitivity of the detection method is reduced [data not shown]. Data presented here show that there was no evidence of PCR inhibitors co-purified using either ME or EZ methods. In some aspects, use of 1 μl of DNA template provides a highly sensitive method of detection for the at least one Plasmodium ssp.

In some aspects, the disclosure demonstrates that low volume reactions were more sensitive, with 2 μl total reaction volume being sensitive FOR Plasmodium detection. In some aspects, it was found that when the reaction master mix used in qPCR was titrated, there was a negative correlation between total reaction volume and qPCR sensitivity. Not to be bound by theory, but it may be that with small volumes, the temperature cycling is more efficient.

In some aspects, the present methods can be carried out in total reaction volumes of from about 2 μl to about 20 μl. In some aspects, the reaction volume can be 2 μl, 3 μl, 4 μl, 5 μl, 6 μl, 7 μl, 8 μl, 9 μl, 10 μl, 11 μl, 12 μl, 13 μl, 14 μl, 15 μl, 16 μl, 17 μl, 18 μl, 19 μl or 20 μl. Lower reaction volumes can lower cost of performance of the methods of the present disclosure, which allows the method to be practiced in regions in which cost may be prohibitive of using a PCR method. Both reaction volume and the concentration of the reactants are critical to ensure success of a low volume qPCR reaction.

Cost is one of the most prohibitive aspects of qPCR especially in resource constrained laboratories in austere locations where malaria is found. As such, cost has inhibited the adoption of qPCR over microscopy as the gold standard method for malaria diagnosis. Commercial master mix kits, for example, QuantiFast Probe PCR recommends using a total reaction volume of 20-25 μl. Excluding the cost of DNA extraction and labor, at the current list prices of master mixes, primers and probes, a singleplex reaction containing total volume of 25 μl costs ˜$1 whereas a reaction containing total volume of 3 μl costs ˜$0.08, more than 90% reduction in cost. A four target multiplex reaction as described here costs ˜$0.11. The reduced costs of qPCR as described makes its application in high through-put qPCR methods advantageous for epidemiological and surveillance studies. It is important to note however that the cost of DNA extraction remains the single most limiting factor. It is extremely important that less expensive DNA extraction methods are developed for PCR to be more affordable and accessible. In this study, DNA was extracted using column based methods. However, large scale field studies often employ less expensive and simpler methods of DNA extraction such as Chelex-100 extraction. If well stored, extracted DNA can be used in numerous PCR experiments which can be argued that it lowers the cost of DNA extraction.

Suitable samples include, but are not limited to, a blood sample from a subject, DNA extracted from a sample from a patient, purified DNA from a sample.

Suitable subjects include, but are not limited to, a subject or patient that may have been exposed to or infected with one or more strains of Plasmodium; a subject or patient that has been exposed to or infected with one or more strains of Plasmodium; a subject or patient presenting with clinical symptoms of malaria, a subject or patient that has been bitten by a mosquito; a subject or patient that is participating in a study including, but not limited to, studies related to malaria vaccination, new or existing malaria treatments, etc.; a subject or patient that has previously been infected with Plasmodium or is no longer showing symptoms of Plasmodium infection; a subject suspected of being infected with Plasmodium either without clinical symptoms or with one or more clinical symptoms; a subject or patient living in a high malaria area; or a subject or patient involved in clinical investigations. Suitable subjects would be recognized by one skilled in the art. Suitably, subjects or patients are mammals, preferably humans.

Clinical symptoms of malaria are generally known in the art and may include, for example, fever, chills, headache, sweats, fatigue, nausea, vomiting, muscle and/or back pain, dry cough, etc.

Standard PCR techniques are used in the methods of this disclosure. One skilled in the art would appreciate the PCR techniques and PCR machines used in the practice of this disclosure are standard and readily available. A typical PCR reaction includes multiple amplification steps, or cycles that selectively amplify a target nucleic acid species. A full description of the PCR process, and common variations thereof, such as quantitative PCR (QPCR), real-time QPCR, reverse transcription PCR (RT-PCR) and quantitative reverse transcription PCR (QRT-PCR) are well-described in the art and have been broadly commercialized. Suitable PCR techniques can be found in “Current Protocols in Molecular Biology” editor Gwen P. Taylor, 2004, incorporated by reference in its entirety. For a summary of PCR methods used for malaria, see, e.g., Alemayehu et al. Malaria Journal 2013, 12:277, incorporated by reference in its entirety.

A typical PCR reaction includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 25-30 or more cycles of denaturation, annealing and elongation. In many cases, the annealing and elongation steps can be performed concurrently, in which case the cycle contains only two steps.

As discussed above, the procedures described herein also may be used in multiplex quantitative real time (QRT)-PCR processes. In its broadest sense, a multiplex PCR process involves production of two or more amplicons in the same reaction vessel. Multiplex amplicons may be analyzed by gel electrophoresis and detection of the amplicons by one of a variety of methods, such as, without limitation ethidium bromide staining, Southern blotting and hybridization to probes, or by incorporating fluorescent or radioactive moieties into the amplicons and subsequently viewing the product on a gel. However, real-time monitoring of the production of two or more amplicons is preferred. The fluorescent 5′ nuclease assay is the most common monitoring method. Equipment is now available (for example, the below-described cycler and for example, TaqMan products) that permits the real-time monitoring of accumulation of two or more fluorescent reporters in the same tube. For multiplex monitoring of the fluorescent 5′ nuclease method, oligomers are provided corresponding to each amplicon species to be detected. The oligomer probe for each amplicon species has a fluorescent reporter with a different peak emission wavelength than the oligomer probe(s) for each other amplicons species. The accumulation of each unquenched fluorescent reporter can be monitored to determine the relative amounts of the target sequence corresponding to each amplicon.

In traditional multiplex QPCR and QRT-PCR procedures, the selection of PCR primer sets having similar annealing and elongation kinetics and similar sized amplicons are desirable. The design and selection of appropriate PCR primer sets is a process that is well known to a person skilled in the art. A balanced multiplex reaction is preferably, where certain amplicon(s) do not out-compete the other amplicon(s) for resources, such as dNTPs or enzyme. Equalization of the Tm (melting temperature) for all PCR primer sets also is encouraged. See, for example, ABI PRISM 7700 Sequence Detection System User Bulletin #5, “Multiplex PCR with TaqMan VIC Probes”, Applied Biosystems (1998/2001).

In another aspect, the disclosure provides a highly sensitive genus-specific quantitative reverse transcriptase real-time PCT (qRT-PCR) assay for detection of Plasmodium and show that amplification of total nucleic acids (RNA and DNA) of the 18S rRNA genes increases the analytical sensitivity of the assay, as detailed in Example 2.

The methods disclosed herein provide a basis for establishing PCR assays as the gold standard for malaria diagnosis and surveillance, not only in clinical research, but also in future monitoring and evaluation efforts in malaria control and elimination campaigns. The sensitivity of the methods provided herein compare well to the current gold standard, microscopy of thick blood smears.

The presently described technology and its advantages will be better understood by reference to the following examples. These examples are provided to describe specific embodiments of the present technology. By providing these specific examples, the applicants do not limit the scope and spirit of the present technology. It will be understood by those skilled in the art that the full scope of the presently described technology encompasses the subject matter defined by the claims appending this specification, and any alterations, modifications, or equivalents of those claims.

The citations provided herein are hereby incorporated by reference for the cited subject matter.

### Example 1

### Materials and Methods

Ethics

Clinical samples used in this study were obtained either from Kenya [P. falciparum] or Cambodia [P. vivax]. The Kenyan samples were from a Phase IIb pediatric clinical trial conducted between March 2005 and April 2006 at the KEMRI/Walter Reed Project, Kombewa Clinic in the Kombewa Division of Kisumu District, Nyanza Province, Western Kenya. The trial registration for this study can be found at clinicaltrials.gov, identifier NCT00317473. The details of this study have also been published elsewhere[14]. The study was approved by Ethical Review Committee of the Kenya Medical Research Institute, Nairobi, Kenya. The Cambodian samples were from a study conducted in 2010 in Battambang and Oddar Meancheay Provinces along the Thai border. The details of this study have been published elsewhere [15]. This study was approved by the National Ethical Committee for Health Research, Phnom Penh, Cambodia. Both studies were also approved by the Walter Reed Army Institute of Research (WRAIR) Institutional Review Board, Silver Spring, Md., USA and by the Human Subjects Research and Review Board of the Surgeon General of the U.S. Army at Fort Detrick, Md., USA. The Cambodia study was conducted under approved protocol WRAIR 1576. Protocols used in these studies complied with International Conference on Harmonization Good Clinical Practice (ICH-GCP) guidelines. These studies were conducted in accordance with the principles described in the Nuremberg Code and the Belmont Report including all federal regulations regarding the protection of human participants as described in 32 CFR 219 (The Common Rule) and instructions from the Department of Defense and the Department of the Army. They also followed the internal policies for human subject protections and the standards for the responsible conduct of research of the US Army Medical Research and Materiel Command. WRAIR holds a Federal Wide Assurance from the Office of Human Research Protections under the Department of Health and Human Services. All key study personnel in both studies were certified as having completed mandatory human research ethics education curricula and training under the direction of the WRAIR IRB Human Subjects Protection Program. All potential study subjects provided written informed consent before screening and enrollment and had to pass an assessment of understanding.

Clinical Samples

For assessment of malaria, a peripheral blood smear was obtained from subjects who presented to the study sites with fever or a history of fever within 48 h or an illness that the attending doctor suspected might be due to malaria infection. After Giemsa staining, thin and thick blood smear slides from each sample were independently examined by two or three expert microscopists for detection of Plasmodium and counts where applicable. All malaria microscopists were fully trained and were required to pass a competency and proficiency test prior to reading slides for the study. The parasite density presented in this study is the average density obtained by the independent (blinded from each other's results) microscopists. Blood samples obtained from these studies were stored frozen in −20° C. until needed. Genomic DNA was extracted from the whole blood either manually using the QIAamp DNA Blood Mini Kit or automated with the EZ1 DNA blood kit on the EZ1 Advanced XL automated sample purification system (Qiagen, Valencia, Calif.) as recommended by the manufacturer. The DNA from the two studies was extracted at different time points; the DNA from the Cambodian trial was extracted when this study was being conducted, but the DNA from the Kenyan trial was extracted 5-6 years ago. The extracted DNA was stored in −20° C. until needed.

Plasmodium Falciparum Reference Reagent

The WHO international standard for P. falciparum DNA nucleic acid amplification technology (NAT) assays, obtained from the National Institute for Biological Standards and Control (NIBSC; Hertfordshire, United Kingdom) was used as the calibration reference reagent for of the Plasmodium spp. and P. falciparum assays. The standard consists of a freeze-dried preparation of whole blood collected by exchange transfusion from a patient infected with P. falciparum. Following NIBSC recommendations, this lyophilized material was suspended in 500 μl of sterile, nuclease-free water to a final concentration of 1×109 IU/ml, which corresponds to a parasitemia of 9.79 parasites/100 red blood cells [11]. The parasite density of the NAT assays after reconstitution was estimated to be 469,920 parasites/μl, based on the average red blood cell count [from uninfected donor] of 4.8×106 RBC/μl. Unless otherwise indicated, fresh uninfected whole blood was used as a diluent to prepare serial dilutions. The uninfected whole blood was obtained from donors from Washington DC metropolitan area under WRAIR approved protocol. After reconstitution, genomic DNA was extracted with the EZ1 DNA blood kit on the EZ1 Advanced XL automated sample purification system (Qiagen, Valencia, Calif.) as recommended by the manufacturer.

Primer and Probes Design

Primers and probes for detection of Plasmodium spp. and P. falciparum have previously been described[5], [16]. Primers and probes for detection of P. vivax and RNaseP genes were designed using Primer Express 3.0 software (Applied Biosystems, Foster City, Calif.) after the alignment of available GenBank sequences for the P. vivax 18S rRNA gene, accession number AY579418 and human RNaseP gene, accession number NM_{—}001104546.1. Fluorophores chosen for each assay were carefully selected and each combination extensively tested to allow optimal performance of the multiplex assay. Table 1 show primer and probe sequences, fluorophores and the length of primers and probes used in this study. Probes for P. falciparum, P. vivax and RNaseP assays contained minor groove binder (MGB) groups which form stable duplexes with single-stranded DNA targets, allowing shorter probes to be used for hybridization based assays.

TABLE 1

| | | | Size |
|---|---|---|---|
| Primer | SEQUENCES 5′-3′ | Modifications | (bp) |
| PLU F | GCTCTTTCTTGATTTCTTGGATG (SEQ ID NO: 1) | | |
| PLU R | AGCAGGTTAAGATCTCGTTCG (SEQ ID NO: 2) | | |
| PLU P | ATGGCCGTTTTTAGTTCGTG (SEQ ID NO: 3) | CY5-1B | 100 |
| FAL F | ATTGCTTTTGAGAGGTTTTGTTAGTTT (SEQ ID NO: 4) | | |
| FAL R | GCTGTAGTATTCAAACACAATGAACTCAA (SEQ ID NO: 5) | | |
| FAL P | CATAACAGACGGGTAGTCAT (SEQ ID NO: 6) | FAM-MGB | 95 |
| VIV F | GCAACGCTTCTAGCTTAATCCAC (SEQ ID NO: 7) | | |
| VIV R | CAAGCCGAAGCAAAGAAAGTCC (SEQ ID NO: 8) | | |
| VIV P | ACTTTGTGCGCATTTTGCTA (SEQ ID NO: 9) | V1C-MG8 | 133 |
| RNaseP F | TGTTTGCAGATTTGGACCTGC (SEQ ID NO: 10) | | |
| RNaseP R | AATAGCCAAGGTGAGCGGCT (SEQ ID NO: 11) | | |
| RNaseP P | TGCGCGGACTTGTGGA (SEQ ID NO: 12) | NED-MG8 | 84 |
| IC F | AAAGAAACTAGGAGAGATGTGGAACAA (SEQ ID NO: 13) | | |
| IC R | AGCTTGGCAGCTTTCTTCTCA (SEQ ID NO: 14) | | |
| IC P | ACTGCAGCAGATGACAAGCAGCCCT (SEQ ID NO: 15) | CY3-18 | 75 |

Primers and Probes Sequences Used for qPCR Assays in this Study.

Primer and probes for amplification of Plasmodium spp. P. falciparum. P. vivax, RNaseP and internal control (IC) plasmid DNA assays. Sequences for Forward (F), Reverse (R) primers and the Probe (P) are shown.

Real-Time PCR Assays

Amplification and qPCR measurements were performed using the Applied Biosystems 7500 Fast Real-Time PCR System, v 2.0.5 software. The thermal profile used for qPCR if as follows: 5 min at 95° C.; 40 cycles of 3 s at 95° C.; 30 s 60° C. Each reaction contained 1 μL of template DNA and a reaction master mix containing 1× QuantiFast Probe PCR Master Mix with ROX dye (QIAGEN, USA), 0.4 μM of each primer and 0.2 μM of each probe. All qPCR assays were run with appropriate controls including the Non-Template Control [NTC]. If the assay did not contain DNA or the DNA was below the detection limit, the assay result is denoted as ‘und’ [undetermined].

Generation of Plasmid DNAs

Primers for Plasmodium spp. (PLU), P. falciparum (FAL) and P. vivax (VIV) assays were used for amplification of PCR fragments from genomic DNA from either P. falciparum 3D7 laboratory strain samples or P. vivax clinical samples and cloned into TOPO TA vectors. These plasmids are referred to as PLU, FAL or VIV plasmid. To create an inhibition control [IC] plasmid, part of mouse high mobility group protein (HMGB) was cloned into TOPO TA vector. The details of the cloning process and conditions have been previously described [17]. After plasmid DNA carrying the correct clone was purified and tested, the concentration and purity of plasmid DNA was measured using NanoDrop 2000 (Thermo Fisher Scientific Inc, USA) following the manufacturer's instructions. All DNA samples were required to have a 260/280 ratio of between 1.8 and 2.0. The GE for each assay was calculated using the following equation:

(X g/μL DNA/[nucleotide transcript length×660])×6.022×10²³=Y DNA molecules/μL.

For absolute quantification by qPCR, each plasmid DNA was serially diluted and used in subsequent experiments.

Relative Standard Curves

Genomic DNA from P. falciparum [NAT assays] and P. vivax clinical samples were used to generate the relative standard curves for qPCR. For the P. vivax clinical samples, expert microscopists determined the parasite density. Five different clinical samples were used to generate relative standard curves for qPCR. Genomic DNA from these samples was extracted using the QIAamp Blood DNA kit (Qiagen, Valencia, Calif.), serially diluted and used in the relative quantification experiments.

Results:

Design and Analysis of Multiplex qPCR

A multiplex qPCR assay was designed to simultaneously detect Plasmodium spp., P. falciparum, P. vivax and human RNaseP gene as an endogenous control. These assays are referred to as follows in the manuscript: the Plasmodium spp. assay is referred to as PLU assay, the P. falciparum assay as FAL assay, and the P. vivax as VIV assay. The performance of PLU and FAL assays has been previously described [5]. The sensitivity and specificity of VIV assay was tested using field clinical samples with known parasite densities. To test the analytical sensitivity of the VIV assay, P. vivax clinical samples were analyzed using previously published nested PCR assay [18] and then sequenced using standard methods. All nested PCR results and sequences were that of P. vivax. To test the specificity of the VIV assay, qPCR experiments were performed using the following non-target agents: P. ovale, P. malariae, P. cynomolgi, P. knowlesi, Babesia microti, Trypanosoma cruzi and Leishmania. The VIV assay did not cross-react with any of the non-target organisms tested indicating that the VIV assay has 100% specificity. To test and analyze the assays as a multiplex, genomic DNA containing both P. falciparum and P. vivax, serially diluted 5-fold to 4 different concentrations was used. The performance of each primer and probe set as a singleplex assay (reaction master mix containing a single set of the primer and probe) and multiplex assay (reaction master mix containing primer and probe sets for all the four targets) was assessed. For the multiplex reactions, analysis was performed for individual targets as well as simultaneous analysis of all the targets. One of the most important features of ABI 7500 system is the ability to scan all the wavelengths during the run and stores this information. After the run is complete, different fluorophores can be selected and re-analyzed. This feature permitted us to run multiplex assays but go back and analyze these assays as multiplex or singleplex. Table 2 shows the average CT values of 5-fold serially diluted genomic DNA, each assay performed in 4 replicates. Data shows that all singleplex and multiplex assays performed the same except for RNaseP assay which performed better in analysis 2. There is no good explanation for this phenomenon since the performance of RNaseP assay is the same for analysis 1 and analysis 3.

TABLE 2

| Sample Name | Assay Performed | Analysis 1 Mean CT | Analysis 2 Mean CT | Analysis 3 Mean CT |
|---|---|---|---|---|
| DNA dilution #1 | PLU | 19.8 | 19.85 | 19.92 |
| DNA dilution #2 | PLU | 22.03 | 22.13 | 22.01 |
| DNA dilution #3 | PLU | 24.38 | 24.43 | 24.33 |
| DNA dilution #4 | PLU | 26.73 | 26.78 | 26.79 |
| DNA dilution #1 | FAL | 21.93 | 21.77 | 22.08 |
| DNA dilution #2 | FAL | 24.28 | 24.19 | 24.6 |
| DNA dilution #3 | FAL | 26.32 | 26.3 | 27.01 |
| DNA dilution #4 | FAL | 29.62 | 29.49 | 30.06 |
| DNA dilution #1 | VIV | 22.17 | 22 | 22.22 |
| DNA dilution #2 | VIV | 24.9 | 24.63 | 24.96 |
| DNA dilution #3 | VIV | 22.39 | 27.08 | 27.46 |
| DNA dilution #4 | VIV | 30.27 | 29.94 | 30.05 |
| DNA dilution #1 | RNaseP | 25.81 | 22.5 | 25.24 |
| DNA dilution #2 | RNaseP | 27.79 | 24.98 | 27.62 |
| DNA dilution #3 | RNaseP | 29.98 | 27.38 | 30.29 |
| DNA dilution #4 | RNaseP | 33.92 | 30.11 | 32.75 |

Multiplex qPCR assays were performed containing both primer and probe sets for all four targets or primer and probe set for single target.
Analysis 1 shows data from mutiplex assay analyzed as mutiplex where all four targets were analyzed simultaneously.
Analysis 2 shows data from mutiplex assay but data was analyzed as a single assay for each target.
Analysis 3 shows data from single assays.
doc10.1371/journal.pone.0071529.t002

Analysis of the Multiplex Real-Time PCR Assay.

Comparison of DNA Extraction Methods and Sample Volume

Genomic DNA was extracted from whole blood either manually using the QIAamp DNA Blood Mini Kit (ME) or automated using the Qiagen EZ1 DNA blood kit (Qiagen, Valencia, Calif.) on the EZ1 Advanced XL automated sample purification system (EZ). Extraction procedures were performed as recommended by the manufacturer. Samples used in these experiments were prepared by adding P. falciparum and P. vivax clinical sample into uninfected fresh whole blood. Genomic DNA was extracted from four different volume ranges of whole blood samples, 200, 100, 50 and 200 μl and was eluted in 200, 100, 50 and 50 μl of elution buffer respectively (referred to as experiments 1, 2, 3 and 4 respectively). Phosphate Buffered Saline (PBS) buffer was added to samples that contained less than 200 μl whole blood to bring the final volume to 200 μl as recommended by the manufacturer. Extraction procedure for each volume being tested was performed in duplicate for both ME and EZ methods. Genomic DNA samples (from each of the duplicate extraction) were analyzed in 4 replicates using multiplex qPCR assay. The mean CT values for PLU assay using DNA extracted by ME or EZ methods were 20.69±0.05, 20.36±0.07, 20.43±0.07, 19.32±0.07 and 20.71±0.07, 20.43±0.09, 20.32±0.08 and 18.95±0.03 for experiments 1, 2, 3 and 4 respectively. Both extraction procedures performed equally well for all four different blood volumes tested. As expected, experiment 4, where genomic DNA was extracted from 200 μl whole blood and eluted in 50 μl elution buffer produced CT values that were lower [indicating more template DNA present] compared to the other experiments. For the convenience of sample processing and quantification, genomic DNA used in all the experiments from this point on was extracted using EZ method from 200 μl whole blood and eluted in 200 μl elution buffer.

Inhibition Studies

Inhibition studies were performed to test and compare the co-purification of PCR inhibitors in samples extracted from whole blood using ME or EZ methods. Real-time PCR experiments were performed as described in the materials and methods section using IC plasmid as the template [with IC F/R primers and IC probe; Table 1] with the following modifications. Each 5 μl reaction contained 1.4 μl of genomic DNA extracted from whole blood using ME or EZ methods and 1 μl of IC plasmid DNA as the template. IC plasmid DNA was tested in two different concentrations, 4 replicates each. Control experiments did not contain genomic DNA sample in the reaction. The mean and standard deviation (STDEV) CT values of all experiments were analyzed. Data shows that there were no differences in performance of the qPCR assay between conditions tested (Table 3). At higher IC plasmid DNA concentration, the mean CT value for experiments containing genomic DNA extracted using ME, EZ methods or control (experiment without extracted genomic DNA) was 23.46±0.26 and at lower IC plasmid DNA concentration, the mean CT value was 28.79±0.23. This data illustrates that extraction of genomic DNA from whole blood sample does not co-purify with substances that inhibit qPCR at the volume tested.

TABLE 3

| | ME | ME | EZ | EZ | Control | Control |
|---|---|---|---|---|---|---|
| | High | Low | High | Low | High | Low |
| Mean CT | 23.4 | 28.86 | 23.65 | 28.92 | 23.32 | 28.5 |
| STDEV | 0.293 | 0.071 | 0.111 | 0.074 | 0.265 | 0.141 |

Two different concentrations of IC DNA were used as the DNA template in the qPCR reactions to test the co-purification of PCR inhibitors in samples extracted from whole blood using ME or EZ methods; High DNA concentration or Low DNA concentration. Control experiments did not contain genomic DNA in the reaction. Each column shows the method which the genomic DNA present [or not for the controls] was extracted [ME or EZ] and amount of IC plasmid present [High or Low].
doi:10.1371/journal.pone.0071539.t003

### Inhibition Studies to Test the Co-Purification of PCR Inhibitors.

Determination of Most Optimal Reaction Volume Required for qPCR Assay

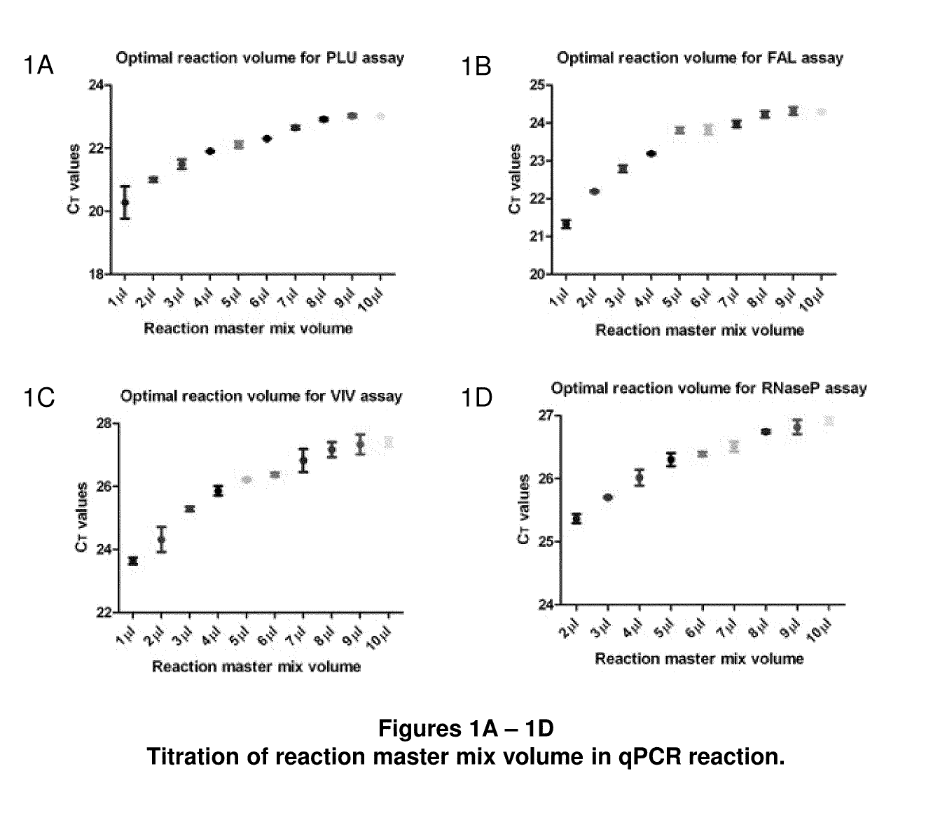

In our previous study [5], qPCR assay was performed by adding 1 μl of template DNA to 9 μl of reaction master mix. The reaction master mix was prepared to a final volume of 20 μl or multiples thereof as needed. To further investigate if the volumes of reaction master mix could be further optimized, a volume titration was performed starting at 10 μl to 1 μl reaction master mix with 1 μl of template DNA used in each reaction. The template DNA used in these experiments contained P. falciparum and P. vivax genomic DNAs. This sample was prepared by mixing P. falciparum and P. vivax clinical sample into uninfected fresh whole blood which was then extracted as described using EZ method. Real-time PCR experiments were performed in replicates of 4, and repeated on two separate occasions bringing the number of total replicates performed to 8. All the four targets in the multiplex qPCR assay were analyzed. Surprisingly, for PLU, FAL and VIV assays, the 1 μl reaction master mix (2 μl total reaction volume) was the most efficient with exception of RNaseP assay which did not work (FIG. 1). The 2 μl reaction master mix assays performed superiorly as well, with an overall CT values slightly better than the rest of the reactions. In general, data showed a trend whereas the reaction volume increased, qPCR assays became slightly less efficient for all the assays with a plateau being reached at reaction master mix of 8 μl. The amplification plots of all the reactions were smooth and looked similar in all the different reaction volumes tested. To further test the importance of molar concentrations of the reactions, starting at 5 μl down to 1 μl reaction master mixes, water was added to bring the final reaction master mix to 10 μl. One microliter DNA template was used in each reaction. Real-time PCR assays were completely compromised with most of the reactions failing to amplify (data not shown).

Performance of the Absolute qPCR Assays

PLU, FAL and VIV plasmids were used to determine the performance of each absolute qPCR assay. The efficiencies and precision of each replicate assay was evaluated. To determine the efficiency, each plasmid DNA was 5-fold serially diluted 5 times and analyzed in 3 replicates. The slope and the R2 values of each curve were used to evaluate the efficiency of each assay whereas STDEV of each replicate was used to evaluate the assay precision. All the absolute qPCR assays performed with efficiency of more than 94%, R2 values were 0.99 or greater and the STDEV of each replicate was <0.167.

Quantification of Absolute qPCR Assay in Terms of Parasite/μl

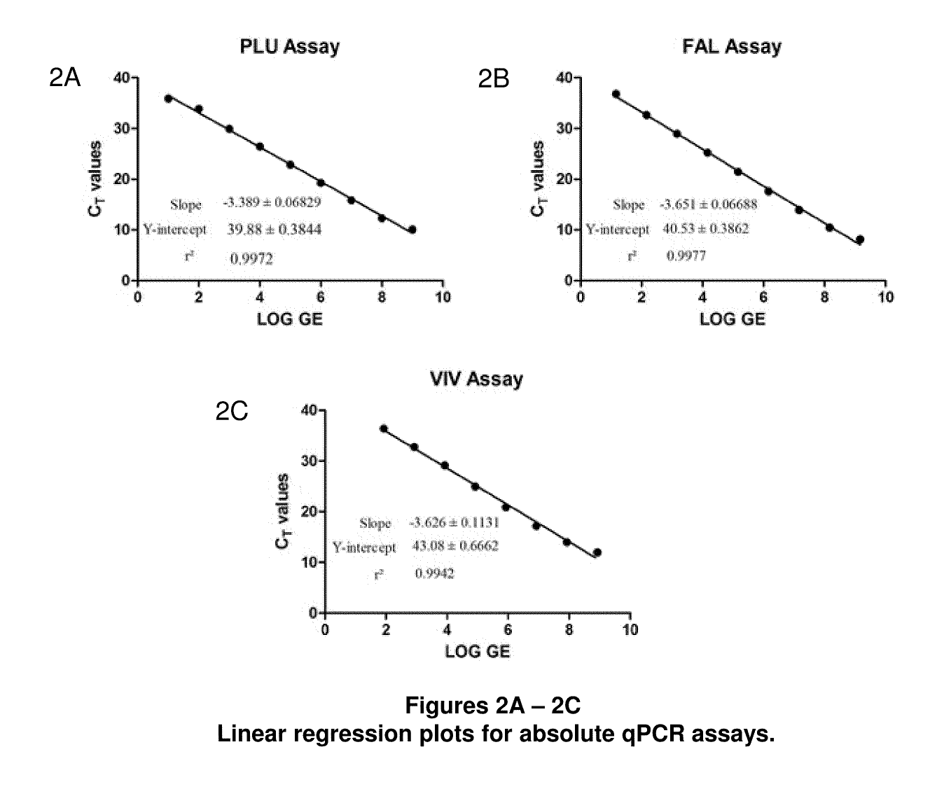

In absolute quantification, sample concentration is expressed in terms of genomic equivalence (GE) or copy numbers. However, for malaria, parasite density is mostly expressed as parasite/μl, based on parasite density as determined by microscopy. It is important therefore that when performing absolute qPCR for malaria, parasite density is expressed in terms that makes clinical sense [and/or other application] and is based on the gold standard for malaria diagnosis which is microscopy. Here, an objective was laid out to determine the amount of GE that is equivalent to parasites/μl. The CT values obtained from absolute and relative qPCR assays were correlated to determine the amount of GE [plasmid DNA] that is equivalent to parasite/μl. NAT assays was used for relative quantification of Plasmodium spp. and P. falciparum assays whereas P. vivax clinical samples were used for relative quantification of the P. vivax assay. The parasite density of the NAT assays was determined as described above. For analysis of P. vivax parasite density using the VIV assay, 5 clinical samples with known parasite densities as determined by expert microscopists were used. Real-time PCR assays were performed for all the three assays using either serially diluted plasmid DNA (absolute qPCR, as shown in Table 4) or genomic DNA (relative qPCR). To estimate the amount of GE that is equivalent to parasites/μl from the relative qPCR assay, the CT values obtained from relative qPCR assays were interpolated as unknowns from the linear regression standard curve of the absolute qPCR assays to obtain equivalent GE (FIG. 2). The amount of GE that corresponds to or is equivalent to parasite density in parasites/μl was estimated based on averages obtained from multiple dilutions for NAT assays and 5 P. vivax clinical isolates that had been serially diluted. For the PLU absolute qPCR assay, 10.05 GE corresponds to 1 parasites/μl or 1 GE is equivalent to 0.1 parasites/μl; for the FAL absolute qPCR assay, 3.55 GE correlates to 1 parasites/μl or 1 GE is equivalent to 0.281 parasites/μl; and for the VIV absolute qPCR assay, 7.88 GE corresponds to 1 parasites/μl or 1 GE is equivalent to 0.127 parasites/μl.

Linear Regression Plots for Absolute qPCR Assays.

TABLE 4

| Sample | Avg | Genomic | Sample | Avg | Genomic | Sample | Avg | Genomic |
|---|---|---|---|---|---|---|---|---|
| Name | C_{Y} | Equivalence | Name | C_{Y} | Equivalence | Name | C_{Y} | Equivalence |
| PLU-1 | 10.036 | 1003666666.67 | FAL-1 | 8.109 | 1459878787.88 | VIV-1 | 11.99 | 843992424.24 |
| PLU-2 | 12.267 | 100366666.67 | FAL-2 | 10.454 | 145987878.79 | VIV-2 | 13.972 | 84399242.42 |
| PLU-3 | 15.827 | 10036666.67 | FAL-3 | 13.92 | 14598787.88 | VIV-3 | 17.19 | 8439924.24 |
| PLU-4 | 19.235 | 1003666.67 | FAL-4 | 17.569 | 1459878.79 | VIV-4 | 20.854 | 843992.42 |
| PLU-5 | 22.85 | 100366.67 | FAL-5 | 21.462 | 145987.88 | VIV-5 | 24.952 | 84399.24 |
| PLU-6 | 23.447 | 10036.67 | FAL-6 | 25.224 | 14598.79 | VIV-6 | 29.13 | 8439.92 |
| PLU-7 | 29.907 | 1003.67 | FAL-7 | 28.939 | 1456.88 | VIV-7 | 32.744 | 843.99 |
| PLU-8 | 33.858 | 100.37 | FAL-8 | 32.665 | 145.99 | VIV-8 | 36.389 | 84.40 |
| PLU-9 | 35.882 | 10.04 | FAL-9 | 36.788 | 14.60 | VIV-9 | Und | 8.44 |
| PLU-10 | Und | 1.00 | FAL-10 | Und | 1.46 | VIV-10 | Und | 0.84 |
| NTC | Und | | NTC | Und | | NTC | Und | |

Data showing the mean C_{Y} values obtained from qPCR assays performed using plasmid DNAs. Plasmid DNAs were 10-fold serially diluted 10-log [times] and ran in 4-8 replicates.
doi:10.1371/journal.pone.0071539.t004

Absolute qPCR CT Values Obtained vs. the Genomic Equivalence Used.

Determination of Limit of Detection

To establish the Limit of Detection (LoD), plasmid DNAs for each assay were 5-fold serially diluted and qPCR assays performed in 4 replicates. The lowest concentration of plasmid DNAs that yielded positive test results in all the replicates were set as the initial LoD. The initial LoD was used as the base point for the 2-fold dilution series to determine the actual LoD. Real-time PCR assays for each plasmid DNA were performed in 4 replicates and actual LoD was established from the lowest plasmid DNA that yielded positive test results in all the replicates. The GE LoD for PLU, FAL and VIV assays were 2.5, 7.3 and 8.4 respectively. To determine LoD for each assay in terms of parasite/μl, GE LoD was multiplied with parasite/μl of GE [0.1, 0.281 and 0.127 for PLU, FAL and VIV assays respectively] of plasmid DNA established for each assay. The calculated LoD in terms parasite/μl based on GE LoD were 0.25, 2.04 and 1.07 for PLU, FAL and VIV assays respectively. Similar dilution strategy was used to establish LoD using genomic DNA. NAT assays DNA was used to determine LoD for PLU and FAL assays whereas P. vivax clinical sample DNA was used to determine LoD for VIV assay. The LoDs for PLU, FAL and VIV assays were 0.31, 2.5 and 1.13 parasite/μl respectively. This data demonstrates GE LoD for the three assays compares very well with LoD established using genomic DNA.

Comparison of Parasite Densities (Parasite/μl) Obtained by Absolute qPCR to Microscopy

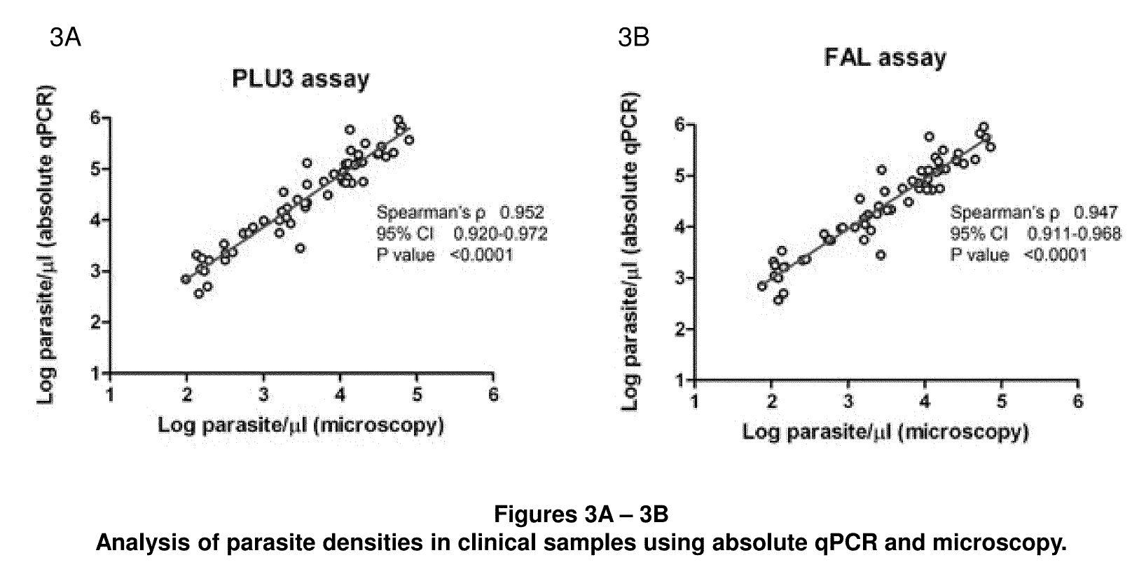

Parasite densities in terms of parasite/μl were determined in 60 clinical samples (from Kenya) using plasmid DNA as the standard for PLU and FAL assays. These densities were then compared to parasite densities obtained by microscopy. There was statistically significant correlation between parasite densities measured by both methods (FIG. 3). The average log 10 density obtained by microscopy was 4.41 whereas for PLU and FAL assays were 3.46 and 3.54 respectively. We did not have sufficient P. vivax samples with well characterized microscopy data to perform similar experiments for the VIV assay.

Conclusion:

Example 1 demonstrates a multiplex assay which absolute quantification of malaria parasite is described. The parasite quantity is described in parasite/μl, the same way as described when quantified by microscopy or relative qPCR. The multiplex assay described here can be used as is in areas where both P. falciparum and P. vivax co-exist in the population such as South East Asia and some parts of Africa such as Ethiopia. Further, a subset of the assays reported here can be used in African populations, with the P. vivax assay replaced with other relevant diagnostic assays for detection of P. ovale and/or P. malariae.

### Example 2

### Materials and Methods

Samples.

Samples used in this study were obtained from a Phase IIb pediatric clinical trial conducted between March 2005 and April 2006 at the KEMRI/Walter Reed Project, Kombewa Clinic, in the Kombewa Division of Kisumu District, Nyanza Province, Western Kenya. The study was approved by the Ethical Review Committee of the Kenya Medical Research Institute, Nairobi, Kenya, and the Walter Reed Army Institute of Research Institutional Review Board, Silver Spring, Md.

The details of this study have been described elsewhere (23). Briefly, EDTA-treated blood samples were collected from study participants at enrollment (day 0) and 1 month after administration of the third and final vaccination. In addition, blood was also drawn during unscheduled clinical visits from children who were sick and suspected to have malaria. For assessment of malaria, a peripheral blood smear was obtained from subjects who presented to the Walter Reed Project's Kombewa Clinic with fever or a history of fever within 48 h or an illness that the attending doctor suspected might be due to malaria infection. After Giemsa staining, thin and thick blood smear slides from each sample were independently examined by three expert microscopists for detection of Plasmodium and counts where applicable. All malaria microscopists were fully trained and were required to pass a competency and proficiency test prior to reading slides for the study. Detection of asexual parasitemia of >0 parasites/μl resulted in the diagnosis of and treatment for malaria. The parasite density presented in this study is the average density obtained by the three independent (blinded from each other's results) microscopists. Two hundred microliters of blood was aliquoted and stored at −20° C. until it was required. Genomic nucleic acid was extracted from whole blood using the QIAamp DNA Blood Mini Kit (Qiagen, Valencia, Calif.) as recommended by the manufacturer. Extracted nucleic acids (NA) were stored at −20° C. until they were required.

Primer and Probe Design.

Primer and probe sets were based on 18S rRNA sequences deposited in GenBank and were designed using the Web-based software Primer3 v.0.4.0 (frodo.wi.mit.edu/primer3/) and/or Primer Express Software (Applied Biosystem, Foster City, Calif.). The Plasmodium genus primers and probe were designed to amplify all units of rRNA distributed in all the chromosomes: 1, 5, 7, 11, and 13. They were also designed to amplify the two types of Plasmodium 18S rRNA genes, the S type, expressed primarily in the mosquito vector, and the A type, expressed primarily in the human host (8, 22). The regions of sequences selected were highly conserved and found only in the genus Plasmodium. The sequence of the forward primer was 5′-GCTCTTTCTTGATTTCTTGGATG-3′ (SEQ ID NO: 1), and that of the reverse primer was 5′-AGCAGGTTAAGATCTCGTTCG-3 (SEQ ID NO: 2)′. The probe sequence was 5′-ATGGCCGTTTTTAGTTCGTG-3′ (SEQ ID NO: 3), labeled with 5′FAM (6-carboxyfluorescein) and 3TAMRA (6-carboxytetramethyl-rhodamine) as the reporter and quencher, respectively. For the P. falciparum species-specific primers and probe, we used previously published sequences but used VIC instead of FAM as the reporter dye (18, 20).

qRT-PCR and qPCR.

Amplification and real-time measurements were performed in the Applied Biosystems 7500 analytical PCR system with the following thermal profile for qPCR: 10 min at 95° C., 40 cycles of 15 s at 95° C., and 1 min at 60° C. For qRT-PCR, a 30-min cycle at 50° C. was added as the initial step for the reverse transcription process. For the reaction, 1 μl of template was added to 9 μl of reaction master mix containing 1× QuantiTect Probe RT-PCR Master Mix (Qiagen), 0.4 μM each primer, 0.2 μM probe, and 4 mM MgCl2. For the qRT-PCR assay, QuantiTect RT Mix (a blend of Omniscript and Sensiscript Reverse enzymes) was added to the reaction master mix as recommended by the manufacturer at a rate of 1 μl per 100 μl of the reaction master mix.

Standard Curves.

For standard curves, cultured highly synchronized ring stage 3D7 parasites were used in order to emulate infected human blood samples. The percent parasitemia of the ring stage was determined by flow cytometry and microscopy. To determine the number of parasites/μl in culture material, we multiplied the percent parasitemia by the number of red blood cells (RBCs)/μl, which were counted by Coulter analysis (Coulter AC•T 5 diff CP; Beckman Coulter, Inc., Miami, Fla.). The limit of detection (LOD) for the PCR assays was established by creating a standard curve using cultured synchronized ring stage 3D7 parasites that were serially diluted using uninfected whole blood prior to total NA extraction. When analyzing and quantifying clinical samples, each 96-well plate was run with the standard 3D7 NA, which was serially 5-fold diluted from 20,000 parasites to 0.256 parasite/μl. Total nucleic acid (RNA and DNA) was extracted using a QIAamp DNA Blood Mini Kit with 200 μl of blood (or cultured material) and eluted in 200 μl of water. For each experiment, we used 1 μl of NA template, which is equivalent to 1 μl of blood from a patient or cultured material.

Statistical Analysis.

For statistical analysis, a two-tailed paired t test in Graph Pad prism was used.

Comparison of Limits of Detection Between qRT-PCR and qPCR Assays.

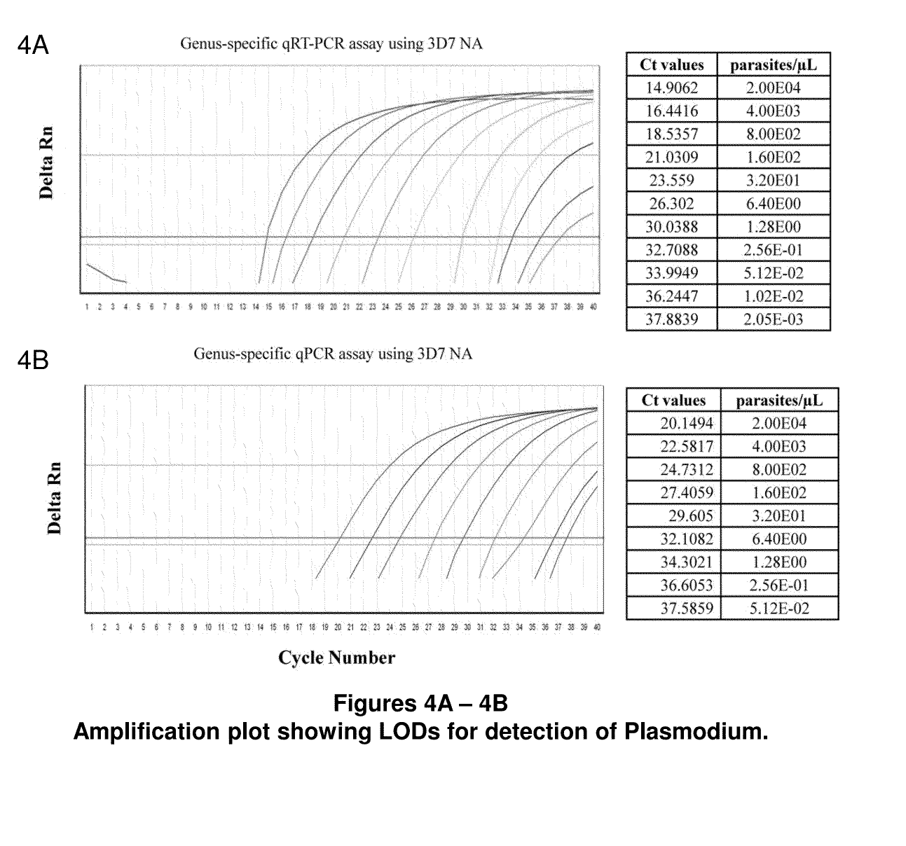

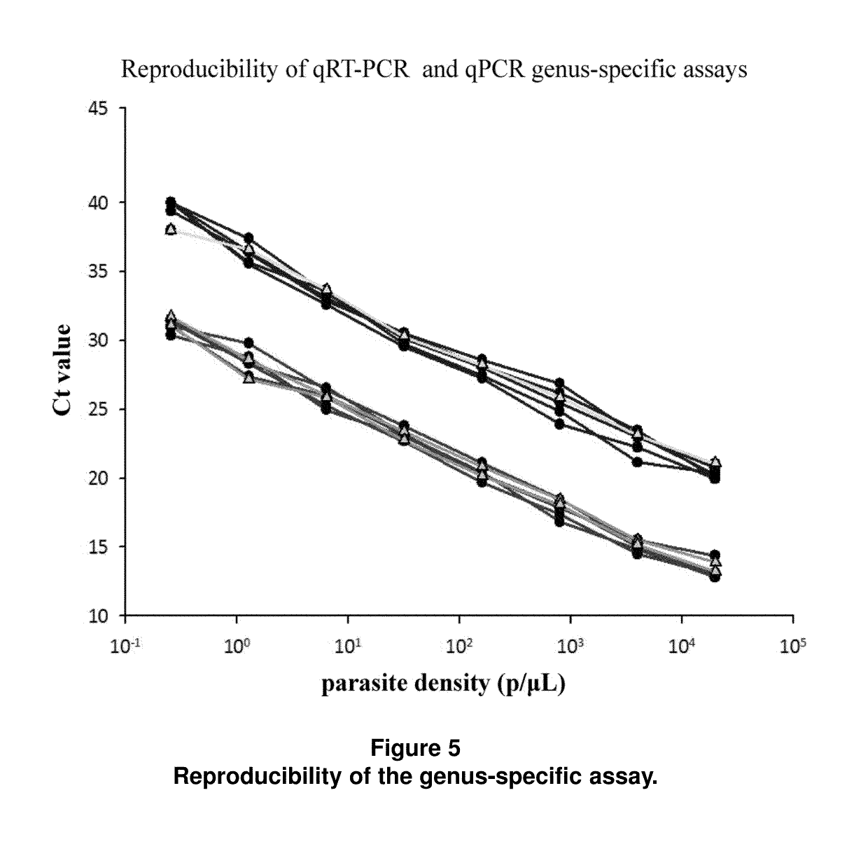

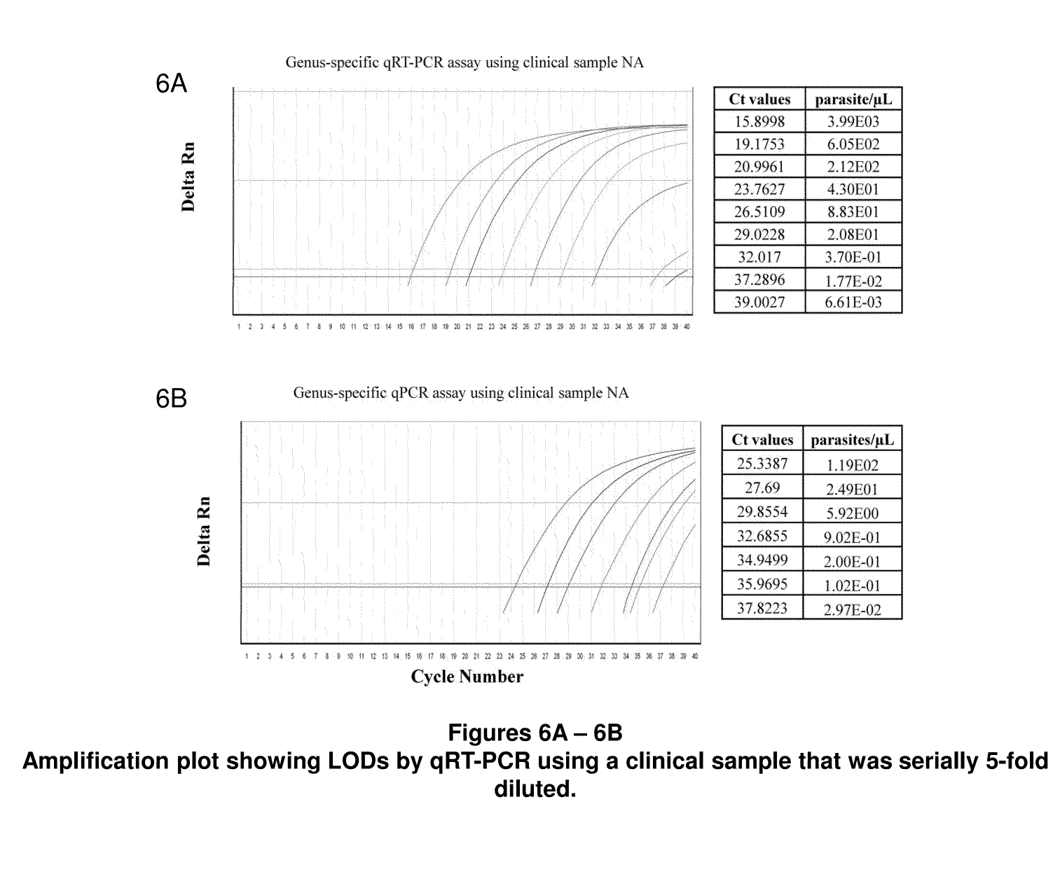

Standard 3D7 NA was used to establish the LOD, which was set as the lowest NA concentration at the threshold cycle number (CT) at which the normalized reporter dye emission rose above background noise. For the genus-specific qRT-PCR assay, the LOD was determined to be 0.002 parasite/μl, and the LOD was 0.0512 parasite/μl for the qPCR assay (FIGS. 4a and b). For the P. falciparum species-specific assay, the LOD was determined to be 1.22 parasites/μl for qRT-PCR and 2.44 parasites/μl for qPCR (data not shown). We assessed the reproducibility of the qRT-PCR and qPCR genus-specific assays with respect to both intra- and interoperator variability on replicate samples conducted on different days. The qRT-PCR assay was found to be more sensitive over a wide dynamic range of known parasite densities than the qPCR assay. Both assays were highly reproducible, with a mean coefficient of variation of 3% between different operators performing assays on different days (FIG. 5). Next, we randomly picked a clinical sample that had been established to be P. falciparum positive by microscopy and assessed the LOD for both genus-specific and P. falciparum species-specific assays by serially diluting the sample. The LOD for the genus-specific assay was established to be 0.00661 parasite/μl for qRT-PCR and 0.0297 parasite/μl for qPCR (FIGS. 6A and B). For the P. falciparum species-specific assay, the LOD was established to be 1.82 parasites/μl for qRT-PCR and 3.41 parasites/μl for qPCR (data not shown). We tested the specificities of the assays by ensuring the assays did not amplify human NA.

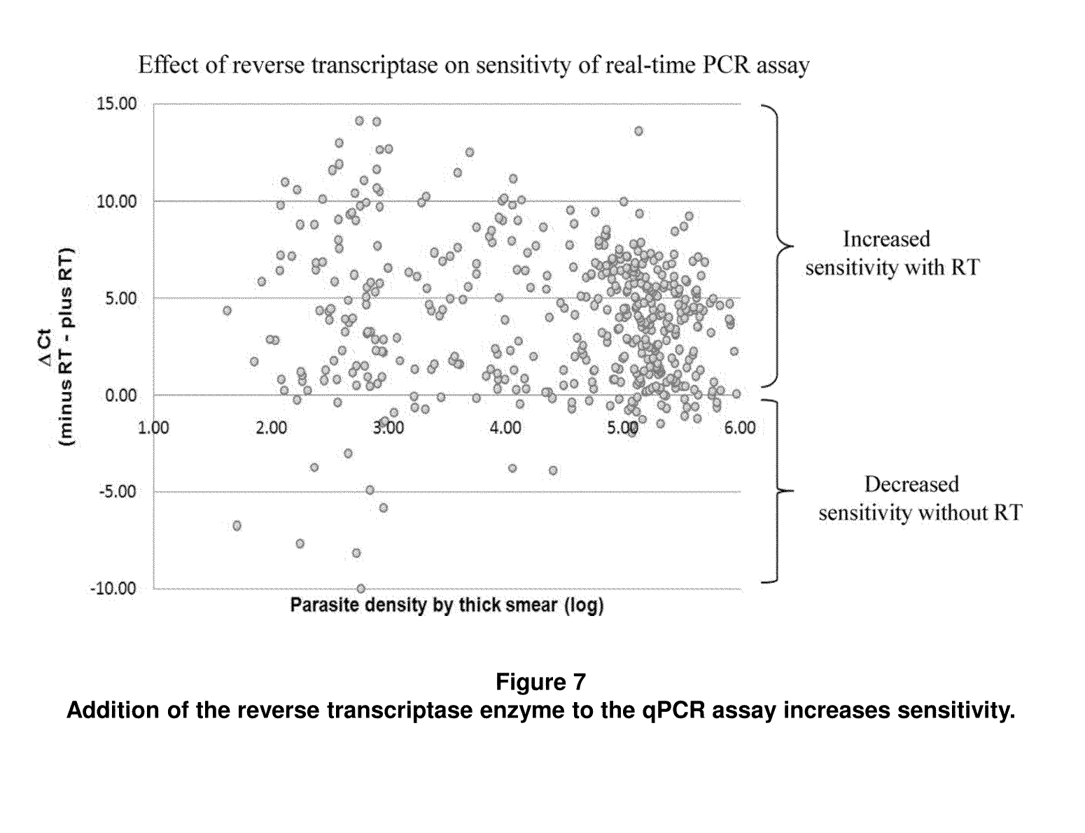

Establishing assay sensitivity by inclusion of a reverse transcriptase step in clinical samples. We then compared the performance of qRT-PCR and qPCR in 603 clinical samples using genus-specific or P. falciparum species-specific assays in a paired t test. There was a significant difference in the performance of qRT-PCR and qPCR for both genus-specific and P. falciparum-specific assays. For the genus-specific assay, the CT values for qRT-PCR and qPCR were significantly different from each other (P<0.0001), with means±standard errors of the mean (SEM) of 17.69±0.2393 and 22.44±0.2373, respectively. The difference between the mean CT values for the qRT-PCR and qPCR assays was 4.757±0.3370. For the P. falciparum species-specific assay, the CT values for qRT-PCR and qPCR were significantly different from each other (P<0.0001), with means±SEM of 25.27±0.2564 and 27.12±0.2343, respectively. The difference between the mean CT values for qRT-PCR and qPCR was 1.756±0.3713. To show how inclusion of the RT step in the qPCR assay increased the sensitivity of the assay, the difference in the CT (ΔCT) for each clinical sample between the qRT-PCR assay and the qPCR assay was plotted against the parasite density as determined by a thick blood smear (FIG. 7). Over 5-log-unit differences in parasite density, the inclusion of the reverse transcriptase enzyme in the qPCR assay increased the sensitivity of the assay (samples with net CT values of >0).

Comparison of Quantification by Microscopy to qRT-PCR Quantification.

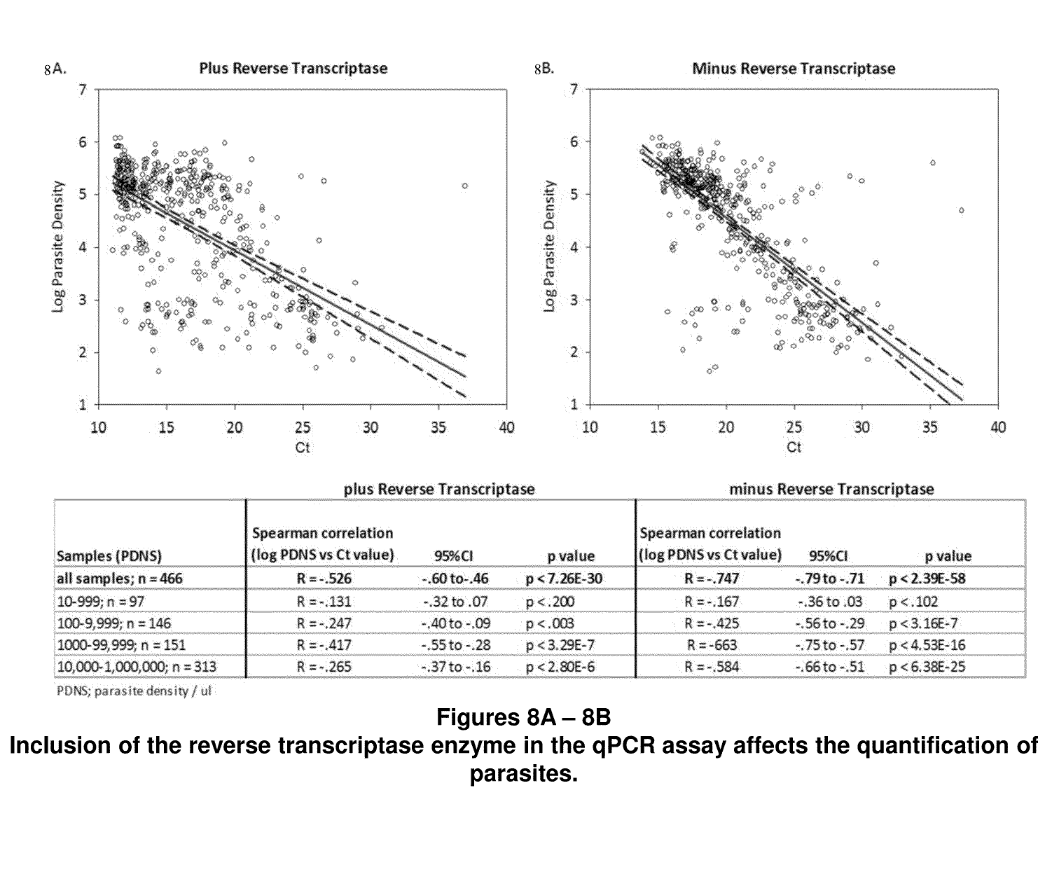

We analyzed clinical samples that had no parasites based on microscopy using genus-specific qRT-PCR assays. Of the 130 samples analyzed, 117 (90%) were positive by qRT-PCR, with a mean CT value of 19.60 and lowest and highest CT values of 11.46 and 39.41. These CT values correspond to quantitative values of 4.65×104 parasites/μl and 0.000362 parasite/μl, respectively. We next determined whether inclusion of the reverse transcriptase enzyme in the qPCR assay affected the quantification of the parasites in the blood over a range of parasite densities (42 to 1.17E6 parasites/μl). From 466 clinical samples, we correlated the parasite density as determined by microscopy with both qRT-PCR and qPCR genus-specific assays (FIG. 8) and measured the statistical significance of each assay for either all samples or samples whose parasite densities were stratified into subgroups. There was a statistically significant correlation between the parasite density measured by microscopy and either the qRT-PCR (FIG. 8A) or qPCR (FIG. 8B) molecular assay. However, the qPCR assay outperformed the qRT-PCR assay for each subgroup examined. The correlation was weakest at low parasite densities in both assays, with increasing divergence of the 95% confidence intervals as the parasite density decreased.

Diluting Clinical Samples Extends the qRT-PCR Dynamic Range.

We observed that at high parasite densities, quantitative PCR reached a plateau, limiting the dynamic range of the qRT-PCR. We hypothesized that the dynamic range of the qRT-PCR can be extended by further diluting the clinical samples. As such, we randomly picked 95 samples with CT values of <18 and performed a 10-fold serial dilution of the NA to 10-4. The diluted samples were analyzed by genus-specific qRT-PCR assay. Before dilution, the mean parasite equivalent as determined by the qRT-PCR assay was 2.09E4 parasites/μl, but after dilution, the mean parasite equivalent increased to 4.33E5 parasites/μl. Interestingly, the mean parasite density of these samples by microscopy was 2.41E5 parasites/μl, clearly showing that dilution of extracted NA correlates well with microscopy at high parasite densities. These data represent more than 1 log unit increase in the number of parasites detected by qRT-PCR after diluting the clinical samples, proving our hypothesis to be true.

1. A method of quantifying the amount of one or more Plasmodium species in a sample comprising:
(a) amplifying in parallel:
(i) a target nucleic acid sequence, when present in the sample, from the one or more Plasmodium species;
(ii) a standard nucleic acid sequence corresponding to the one or more Plasmodium species, and having a predetermined concentration;
(b) detecting the amount of (i) the target nucleic acid sequence and (ii) the standard nucleic acid sequence, each amplified in (a); and
(c) quantifying the amount of the amplified target nucleic acid sequence by correlating the amount of the amplified target nucleic acid sequence detected in (b) to the amount of the amplified standard detected in (b).

2. The method of claim 1, wherein the step (a)(ii) comprises amplifying at least two different predetermined concentrations of the standard nucleic acid sequence.

3. The method of claim 1, wherein the step (a)(ii) comprises amplifying at least three different predetermined concentrations of the standard nucleic acid sequence.

4. The method of claim 1, wherein step (a)(i) and (a)(ii) comprise amplifying in parallel the target nucleic acid sequence and the standard nucleic acid sequence using a primer pair specific for Plasmodium ssp., wherein the primers include a forward primer SEQ ID NO: 1 and reverse primer SEQ ID NO: 2.

5. The method of claim 1, wherein step (a)(i) and (a)(ii) comprise amplifying in parallel the target nucleic acid sequence and the standard using a primer pair specific for P. falciparum, wherein the primers include a forward primer SEQ ID NO: 4 and reverse primer SEQ ID NO: 5.

6. The method of claim 1, wherein step (a)(i) and (a)(ii) comprise amplifying in parallel the target nucleic acid sequence and the standard using a primer pair specific for P. vivax, wherein the primers include a forward primer SEQ ID NO: 7 and reverse primer SEQ ID NO: 8.

7. The method of claim 1, further comprising:
extracting DNA from the sample prior to step (a).

8. The method of claim 7, further comprising:
amplifying RNaseP as an internal control for the DNA extraction process using a primer pair specific for human RNaseP, wherein the primers include a forward primer SEQ ID NO: 10 and reverse primer SEQ ID NO: 11.

9. The method of any of the proceeding claim 1, wherein the standard nucleic acid sequence comprises plasmid DNA.

10. The method of claim 1, wherein the step (a) comprises amplifying in parallel:
(i) a first target nucleic acid sequence from a first Plasmodium species and a second target nucleic acid sequence from a second Plasmodium species;
(ii) a first standard nucleic acid sequence of a predetermined concentration corresponding to the first Plasmodium species and a second standard nucleic acid sequence of a predetermined concentration corresponding to the second Plasmodium species.

11. The method of claim 10, wherein the first and second Plasmodium species are selected from the group P. falciparum and P. vivax.

12. The method of claim 11, wherein step (a)(i) and (a)(ii) comprises amplifying in parallel the target nucleic acid sequence and the standard nucleic acid sequence using a first primer pair specific for P. falciparum, wherein the primers include a forward primer SEQ ID NO: 4 and reverse primer SEQ ID NO: 5, and a second primer pair specific for P. vivax, wherein the primers include a forward primer SEQ ID NO: 7 and reverse primer SEQ ID NO: 8.

13. The method of claim 10, further comprising a probe for each target, wherein each probe comprises a unique reporter molecule that distinguishes the presence of each target present in the sample, and wherein the targets to the at least two Plasmodium species are amplified in the same reaction.

14. The method of claim 13, wherein the probe specific to P. falciparum comprises SEQ ID NO: 6 and the probe specific to P. vivax comprises SEQ ID NO: 9.

15. The method of claim 13, wherein the unique reporter molecule linked to each probe comprises a fluorophore.

16. The method of claim 15, wherein the fluorophore is selected from the group consisting of CY-5, FAM, VIC, NED, and CY3.

17. The method of claim 10, wherein the first and second standard nucleic acid sequences comprise plasmid DNA.

18. The method of claim 10, wherein step (a) further comprises:
(i) a third target nucleic acid sequence from Plasmodium ssp.
(ii) a third standard nucleic acid sequence of a predetermined concentration corresponding to the Plasmodium ssp.

19. The method of claim 18, further comprising a probe for the third target and wherein the three target nucleic acids are amplified in a single reaction.

20. The method of claim 18, wherein the amplifying the third target and the third standard comprises a third primer pair specific for Plasmodium ssp., wherein the third primer pair includes a forward primer SEQ ID NO: 1 and reverse primer SEQ ID NO: 2.

21. The method of claim 10, further comprising:
extracting DNA from the sample prior to step (a).

22. The method of claim 21, further comprising:
amplifying RNaseP as an internal control for the DNA extraction process using a primer pair specific for human RNaseP, wherein the primers include a forward primer SEQ ID NO: 10 and reverse primer SEQ ID NO: 11.

23. The method of claim 22, wherein RNaseP is amplified in the same reaction as the one or more target nucleic acid sequences, wherein the reaction further comprises a probe specific for RNaseP linked to a unique reporter molecule.

24. The method of claim 23, wherein the probe is SEQ ID NO: 12.

25. (canceled)

26. (canceled)

27. The method of claim 1, further comprising the step of
d) converting the amount of the target nucleic acid sequence to a number that indicates the number of plasmodium parasites per microliter (parasites/uL).

28. A method of identifying two or more Plasmodium species in a sample comprising:
(a) amplifying in parallel:
(i) a first target nucleic acid sequence from a first Plasmodium species and a second target sequence from an at least one additional Plasmodium species, when at least two Plasmodium species are present in the sample;
(ii) a first standard nucleic acid sequence and at least one additional standard nucleic acid sequence of a predetermined concentration, wherein the first and at least one additional standards correspond to the first and the at least one additional Plasmodium species of (i), respectively;
(b) detecting the amount of the (i) first and at least one additional target nucleic acid sequence and (ii) first and at least one additional standard nucleic acid sequence amplified in (a); and
(c) determining the presence of the first or the at least one additional Plasmodium species in the sample.

29. The method of claim 28, wherein the first Plasmodium species is P. falciparum and the at least one additional Plasmodium species is P. vivax.

30-47. (canceled)
